(19) United States
(12) Patent Application Publication
(10) Pub. No.: US 2006/0003460 A1
(43) Pub. Date: Jan. 5, 2006

(54) Method for comparing proteomes

(75) Inventors: Ron D. Appel, Meinier (CH); Patricia Palagi, Chene-Bourg (CH)

Correspondence Address: DECHERT LLP, P.O. BOX 10004, PALO ALTO, CA, 94303, US

(73) Assignee: Institut Suisse de Bioinformatique, Geneva (CH)

(21) Appl. No.: 11/189,407

(22) Filed: Jul. 25, 2005

Related U.S. Application Data
(63) Continuation of application No. PCT/IB03/01126, filed on Mar. 25, 2003.

(51) Int. Cl. G01N 33/00

(52) U.S. Cl. 436/86

(57) ABSTRACT

The present invention relates to an improved method for comparing two or more samples containing proteins. This invention consists in first correlating and selecting the huge amount of data generated by mass spectrometry (PMF and MS/MS) before any kind of protein identification process, and then only identifying the proteins that are differentially expressed in dissimilar proteomes and thus have a potentially important biological interest. To do so, the experimental data resulting from separation and mass spectrometry are first correlated according to a correlation method, and then selected according to specific selection criteria. At this stage only, the selected data are analysed to identify the corresponding proteins.

### BACKGROUND OF THE INVENTION

1. Field of the Invention

This invention relates to the field of proteomics and particularly to an improved method for comparing one or more samples containing proteins. More specifically, the method improves the efficiency of identifying proteins that are differentially expressed in different proteomes.

The following references are either cited in the text or relevant to the prior art:
Bafna V. and Edwards N. (2001). SCOPE: a probabilistic model for scoring tandem mass spectra against a peptide database. Bioinformatics Suppl 1, 13-21.
Bairoch, A. and Apweiler, R. (2000). The SWISS-PROT protein sequence database and its supplement TrEMBL in 2000. Nucleic Acids Res. 28, 45-48.
Barker, W. C., Garavelli, J. S., Huang, H., McGarvey, P. B., Orcutt, B. C., Srinivasarao, G. Y., Xiao, C., Yeh, L. S., Ledley, R. S., Janda, J. F., Pfeiffer, F., Mewes, H. W., Tsugita, A., and Wu, C. (2000). The protein information resource (PIR). Nucleic Acids Res. 28, 41-44.
Bartels C. (1990). Fast algorithm for peptide sequencing by mass spectrometry. Biomed. Environ. Mass. Spectrom. 19, 363-368.
Benson, D. A., Karsch-Mizrachi, I., Lipman, D. J., Ostell, J., Rapp, B. A., and Wheeler, D. L. (2002). GenBank. Nucleic Acids Res. 30, 17-20.
Bienvenut W V, Sanchez J-C, Karmime A, Rouge V, Rose K, Binz P-A, Hochstrasser D F. (1999) Analytical Chemistry 71: 4800-4807.
Binz P-A, Müller M, Walther D, Bienvenut W V, Gras R, Hoogland C, Bouchet G, Gasteiger E, Fabbretti R, Gay S, Palagi P, Wilkins M, Rouge V, Tonella L, Paesano S, Rosselat G, Karmime A, Bairoch A, Sanchez J-C, Appel R D, Hochstrasser D F. (1999) Analytical Chemistry 71: 4981-4988.
Chen, T., Kao, M. Y., Tepel, M., Rush, J., and Church, G. M. (2001). A dynamic programming approach to de novo peptide sequencing via tandem mass spectrometry. J. Comput. Biol. 8, 325-337.
Clauser K. R., Hall S. C., Smith D. M., Webb J. W., Andrews L. E., Tran H. M., Epstein L. B., and Burlingame A. L. (1995). Rapid mass spectrometric peptide sequencing and mass matching for characterization of human melanoma proteins isolated by two-dimensional PAGE. Proc Natl Acad Sci USA 92(11), 5072-5076.
Dancik, V., Addona, T. A., Clauser, K. R., Vath, J. E., and Pevzner, P. A. (1999). De novo peptide sequencing via tandem mass spectrometry. J. Comput. Biol. 6, 327-342.
Eddes J. S., Kapp E. A., Frecklington D. F., Connolly L. M., Layton M. J., Moritz R. L., Simpson R. J. (2002). CHOMPER: a bioinformatic tool for rapid validation of tandem mass spectrometry search results associated with high-throughput proteomic strategies. Proteomics September;2(9):1097-103.
Edman, P. (1970). Sequence determination. Mol. Biol. Biochem. Biophys. 8, 211-255.
Eng J. K., McCormack, A. L., and Yates, J. R. 3rd(1994). An approach to correlate tandem mass spectral data of peptides with amino acid sequences in a protein database. J. Am. Soc. Mass Spectrom. 5, 976-989.
Fenyo, D., Qin, J., and Chait, B. T. (1998). Protein identification using mass spectrometric information. Electrophoresis 19, 998-1005.
Fernandez-de-Cossio, J., Gonzalez, J., and Besada, V. (1995). A computer program to aid the sequencing of peptides in collision-activated decomposition experiments. Comput. Appl. Biosci. 11, 427-434.
Fernandez-de-Cossio, J., Gonzalez, J., Betancourt, L., Besada, V., Padron, G., Shimonishi, Y., and Takao, T. (1998). Automated interpretation of high-energy collision-induced dissociation spectra of singly protonated peptides by ‘SeqMS’, a software aid for de novo sequencing by tandem mass spectrometry. Rapid Commun. Mass Spectrom. 12, 1867-1878.
Fernandez-de-Cossio, J., Gonzalez, J., Satomi, Y., Shima, T., Okumura, N., Besada, V., Betancourt, L., Padron, G., Shimonishi, Y., and Takao, T. (2000). Automated interpretation of low-energy collision-induced dissociation spectra by SeqMS, a software aid for de novo sequencing by tandem mass spectrometry. Electrophoresis 21, 1694-1699.
Gatlin, C. L., Eng, J. K., Cross, S. T., Detter, J. C., and Yates, J. R. 3^{rd}, (2000). Automated identification of amino acid sequence variations in proteins by HPLC/microspray tandem mass spectrometry. Anal. Chem. 72, 757-763.
Gonnet G. H. A tutorial Introduction to Computational Biochemistry Using Darwin. 1992. E. T. H. Zurich, Switzerland. Ref Type: Report
Gras, R., Muller, M., Gasteiger, E., Gay, S., Binz, P. A., Bienvenut, W., Hoogland, C., Sanchez, J. C., Bairoch, A., Hochstrasser, D. F., and Appel, R. D. (1999). Improving protein identification from peptide mass fingerprinting through a parameterized multi-level scoring algorithm and an optimized peak detection. Electrophoresis 20, 3535-3550.
Gras R., Gasteiger E., Chopard B., Mëller M., and Appel R. D. New learning method to improving protein identification from peptide mass fingerprinting. 2000. 4th Siena 2D electrophoresis meeting. Ref Type: Conference Proceeding
Gras R. and Muller M. (2001). Computational aspects of protein identification by mass spectrometry. Current Opinion in Molecular Therapeutics 3, 526-532.
Hines W. M., Falick A. M., Burlingame A. L., and Gibson B. W. (1992). Pattern-based algorithm for peptide sequencing from tandem mass spectra of peptides. J. American Society for Mass Spectrometry 3, 326-336.
Ishikawa, K. and Niwa, Y. (1986). Computer-aided peptide sequencing by fast atom bombardment mass spectrometry. Biomed. Environ. Mass Spectrom 13, 373-380.
Johnson, R. S. and Biemann, K. (1989). Computer program (SEQPEP) to aid in the interpretation of high-energy collision tandem mass spectra of peptides. Biomed. Environ. Mass Spectrom 18, 945-957.
Johnson, R. S. and Taylor, J. A. (2000). Searching sequence databases via de novo peptide sequencing by tandem mass spectrometry. Methods Mol. Biol. 146, 41-61.
Mann, M., Hojrup, P., and Roepstorff, P. (1993). Use of mass spectrometric molecular weight information to identify proteins in sequence databases. Biol. Mass Spectrom 22, 338-345.
Mann, M. and Wilm, M. (1994). Error-tolerant identification of peptides in sequence databases by peptide sequence tags. Anal. Chem. 66, 4390-4399.
Moore, R. E., Young M. K., Lee T. D. (2002). Qscore: an algorithm for evaluating SEQUEST database search results. J Am Soc Mass Spectrom April;13(4):378-86
Müller M, Gras R, Appel R D, Bienvenut W V, Hochstrasser D F. (2002) Visualization and analysis of molecular scanner peptide mass spectra. J Am Soc Mass Spectrom March;13(3):221-31.
Pappin D. D. J., Hojrup P., and Bleasby A. J. (1993). Rapid identification of proteins by peptide-mass finger printing. Curr Biol 3, 327-332.
Perkins D. N., Pappin D. D. J., Creasy D. M., and Cottrell J. S. (1999). Probability-based protein identification by searching sequence databases using mass spectrometry data. Electrophoresis 20, 3551-3567.
Pevzner, P. A., Dancik, V., and Tang, C. L. (2000). Mutation-tolerant protein identification by mass spectrometry. J. Comput. Biol. 7, 777-787.
Pevzner;P. A., Mulyukov, Z., Dancik, V., and Tang, C. L. (2001). Efficiency of database search for identification of mutated and modified proteins via mass spectrometry. Genome Res. 11, 290-299.
Sakurai T., Matsuo T., Matsuda H., and Katakuse I. (1984). Paas 3: A computer program to determine probable sequence of peptides from mass spectrometric data. Biomed. Mass Spectrom. 11(8), 396-399.
Siegel, M. M. and Bauman, N. (1988). An efficient algorithm for sequencing peptides using fast atom bombardment mass spectral data. Biomed. Environ. Mass Spectrom. 15, 333-343.

Stoesser, G., Baker, W., van den, B. A., Camon, E., Garcia-Pastor, M., Kanz, C., Kulikova, T., Leinonen, R., Lin, Q., Lombard, V., Lopez, R., Redaschi, N., Stoehr, P., Tuli, M. A., Tzouvara, K., and Vaughan, R. (2002). The EMBL Nucleotide Sequence Database. Nucleic Acids Res. 30, 21-26.
Tateno, Y., Imanishi, T., Miyazaki, S., Fukami-Kobayashi, K., Saitou, N., Sugawara, H., and Gojobori, T. (2002). DNA Data Bank of Japan (DDBJ) for genome scale research in life science. Nucleic Acids Res. 30, 27-30.
Taylor, J. A. and Johnson, R. S. (1997). Sequence database searches via de novo peptide sequencing by tandem mass spectrometry. Rapid Commun. Mass Spectrom. 11, 1067-1075.
Taylor, J. A. and Johnson, R. S. (2001). Implementation and uses of automated de novo peptide sequencing by tandem mass spectrometry. Anal. Chem. 73, 2594-2604.
Traini M, Gooley A A, Ou K, Wilkins M R, Tonella L, Sanchez J-C, Hochstrasser D F, Williams K L. (1998) Electrophoresis 19: 1941-1949.
Wilkins M. R., Gasteiger E., Bairoch A., Sanchez J. C., Williams K. L., Appel R. D., and Hochstrasser D. F. (1999a). Protein identification and analysis tools in ExPASy server. Methods Mol Biol 112, 531-552.
Wilkins M. R., Gasteiger E., Wheeler C. H., Lindskog I., Sanchez J. C., Bairoch A., Appel R. D., Dunn M. J., and Hochstrasser D. F. (1999b). Multiple parameter cross-species species protein identification using Multident—a world-wide web accessible tool. Electrophoresis 19, 3199-3206.
Yates, J. R. 3rd, Eng J. K., and McCormak A. L. (1995). Mining genomes: correlating tandem mass spectra of modified and unmodified peptides to sequences in nucleotide databases. Anal. Chem. 67(18), 3202-3210.
Yates J. R. 3rd, Eng J. K., Clauser K., and Burlingame A. L. (1996). Search of Sequence Databases with Uninterpreted High-Energy Collision-Induced Dissociation Spectra of Peptides. J. American Society for Mass Spectrometry 7, 1089-1098.
Zhang, W. and Chait, B. T. (2000). ProFound: an expert system for protein identification using mass spectrometric peptide mapping information. Anal. Chem. 72, 2482-2489.

2. Description of the Prior Art

Proteomics is the study of the proteins resulting from the expression of the genes contained in genomes. Due to important variations of protein expression between cells having the same genome, there are many proteomes for each corresponding genome. As a result, huge amounts of information are involved, and the study of proteome is even more complex than the study of the genome.

A typical goal of proteomics is to identify the protein expression in a given tissue or cell under given conditions. An additional goal of proteomics is to compare the protein expression in the same tissue, cell or physiological fluid under varying conditions (for example disease vs control), and identify the proteins that are differently expressed.

In recent years, proteomics research has gained importance due to increasingly powerful techniques in protein purification/separation, mass spectrometry and identification techniques, as well as the development of extensive protein and nucleic databases from various organisms (Bairoch et al., 2000; Benson et al., 2002; Stoesser et al., 2002; Tateno et al., 2002).

A traditional method for analyzing proteomes involves separation by 1-D and 2-D polyacrylamide-gel electrophoresis. The 1-D gel method is generally used to achieve a crude separation of cell lysates where the most abundant proteins can be separated and detected. 2-D gel electrophoresis is a more powerful method capable of separating out hundreds of protein spots, where the spot pattern is characteristic of protein expression. Typical separation criteria by gel electrophoresis include electrical charge (isoelectric point-pI) and molecular weight. Gel electrophoresis methods (1-D and 2-D) have nevertheless certain fundamental limitations for screening and identification of proteins. Notably, gel electrophoresis separations are slow and have a limited resolution (i.e. can only distinguish between a limited number of proteins (spots)). In recent years, automation has allowed to manage larger quantities of data resulting from 2-D gel electrophoresis, as exemplified by U.S. Pat. No. 5, 993, 627, U.S. Pat. No. 6, 277, 259, and WO 00/55636.

Higher resolution can be attained by other separation methods such as capillary electrophoresis, gas chromatography, micro-channel networks, liquid chromatography and high-pressure liquid chromatography (HPLC), used in complement to gel electrophoresis or alone. These methods allow the separation of greater numbers of proteins, even in difficult conditions (low sample quantities, small molecular weight, highly basic or hydrophobic proteins . . . ). Separation criteria include electrical charge and molecular weight as in gel electrophoresis, as well as hydrophobicity and other physico-chemical criteria.

After separation, the proteins must be identified, by sequencing or other means. Determining the sequence of amino acid residues in a protein was traditionally accomplished by means of N-terminal Edman degradation (Edman, 1970). Edman sequencing unfortunately requires important quantities of a protein (in the order of 10-100 pmols), which exceed the quantities obtained from most current separation techniques.

Today, most large-scale protein identification procedures use mass spectrometry (MS) data as a starting point rather than Edman degradation. Mass spectrometry accurately determines the molecular mass of the analyzed protein.

Additional information can be obtained by cleaving the protein into smaller peptides before measuring their mass by mass spectrometry. The resulting MS spectrum represents a peptide mass fingerprint (PMF), which is characteristic for each protein and describes the peptide mass value as well as the intensities of the peaks. Cleavage of proteins is usually done by enzymatic means, most commonly by trypsin which cleaves specifically the C-terminal side of arginine or lysine.

There are several identification methods from mass spectrometry data (Gras and Muller, 2001). Identification by peptide mass fingerprint requires a pre-existing protein database, either directly produced or derived from a nucleic database. Identification is done by comparing the experimental masses/spectra obtained by MS (PMF) and the theoretical masses/spectra of virtually digested protein sequences present in the database. The shared masses between the experimental and theoretical spectra are used in a more or less elaborated scoring function to identify the protein. Some tools only count the number of matches, such as PepSea (Mann et al., 1993), PeptideSearch (Mann and Wilm, 1994), PeptIdent/MultiIdent (Wilkins et al., 1999a; Wilkins et al., 1999b), while others use a probabilistic and/or statistic approach, such as MassSearch (Gonnet, 1992), MOWSE (Pappin et al., 1993), MS-Fit (Clauser et al., 1995), Mascot (Perkins et al., 1999), ProFound (Zhang and Chait, 2000). Finally, the algorithm developed by Gras, SmartIdent (Gras et al., 1999; Gras et al., 2000), uses a machine learning approach.

Unfortunately, the PMF method may-not always succeed in giving a reliable identification, for example when the concentration of the protein of interest is low, when only a few peptides are found after the digestion process, or when the protein of interest is insufficiently purified. In addition, post-translational modifications (PTMs) or polymorphisms may significantly modify the peptide masses and impair proper matching. Finally, it is possible that the protein of interest is simply not present in the protein database, and therefore cannot be matched.

In cases where identification is uncertain, one can also use tandem mass spectrometry (MS/MS). MS/MS spectra are obtained after selection of a peptide coming from the peptide mass fingerprint of the protein of interest, subsequent fragmentation of said peptide (for example, by collision with a rare gas), and measurement of the produced fragment masses. Ideally, fragmentation occurs between every amino acid of the peptide, and the masses of two adjacent ionic peaks differ by the mass of one amino acid. In addition to a PMF similar to the one obtained from MS identification, MS/MS data provide information concerning the peptide sequence and allow a more detailed interpretation level than MS spectra alone.

Exploiting the information contained in MS/MS spectra is difficult due to various factors. Notably, the fragmentation process is hardly foreseeable and depends, among other things, on the amount of energy used by the mass spectrometer, on the number and the repartition of the charges carried by the ionic fragment, on its sequence, etc.

Two main identification strategies have been devised to exploit MS/MS data: de novo sequencing followed by sequence matching, and direct spectrum matching with theoretical spectra from an existing database.

De novo sequencing consists in deriving a peptide sequence from the mass differences between the generated MS/MS fragment ions without use of any information extracted from a pre-existing protein or nucleic database. To do so, de novo sequencing uses not only the mass values represented by peaks in the mass spectra, but also their position respective to each other.

Early methods required generating all possible sequences whose masses are similar to the spectrum's parent mass and all the corresponding virtual spectra (Sakurai et al., 1984). The experimental spectrum was then compared and matched with the virtual spectra. Another strategy was to make successive possible extension of sequences (Ishikawa and Niwa, 1986). The sequences are built by successive extension with one or more amino acids. Still another, more sophisticated strategy uses the information lying in the succession of the peaks to make the sequence extensions (Siegel and Bauman, 1988), SEQPEP (Johnson and Biemann, 1989). In this approach, the peptide sequence is built step by step, from the masses differences of “neighbor” peaks in the spectrum. This method can be viewed as the precursor of methods based on graph representation: (Bartels, 1990), (Hines et al., 1992), SeqMS (Fernandez-de-Cossio et al., 1995; Fernandez-de-Cossio et al., 1998; Fernandez-de-Cossio et al., 2000), Lutefisk97 (Taylor and Johnson, 1997; Johnson and Taylor, 2000; Taylor and Johnson, 2001), SHERENGA (Dancik et al., 1999), (Chen et al., 2001). The vertices in the graph are built from the peaks of the spectrum and represent masses of potential fragments. Physico-chemical properties are taken into account to associate a score to each vertex. Whenever two vertices differ by the mass of one or several amino acid, they are connected by an arc. Therefore, each path in the graph represent a possible sequence that can be built from the spectrum. Special algorithms then search the graph for the best paths (i.e. having the highest score built from the vertices score belonging to the path), allowing to determine the most probable sequence or sequences corresponding to the experimental spectrum. Accordingly, de novo sequencing results in one or a limited number of possible amino acid sequences, obtained without any recourse to a protein or nucleic database.

For identification purposes, the sequence(s) (partial or complete) obtained de novo are then used to scan a protein database with a standard alignment software. De novo sequencing is a fairly complex task which requires both good quality spectra and manual verification by a mass spectrometry expert. Accordingly, this approach is not adapted to the huge amounts of data generated by high-throughput settings available today.

The alternative to de novo sequencing is to match the experimental peptide spectra obtained from MS/MS with theoretical spectra derived from pre-existing protein databases. Unlike de novo sequencing, most MS/MS spectra matching tools use only the mass values in the MS/MS spectra—to the exclusion of their respective positions. The method most used today for MS/MS identification is the shared peak count (SPC). The ionic masses of the MS/MS spectrum represent an “ion mass fingerprint”, by analogy with the “peptide mass fingerprint”. The experimental MS/MS spectrum is compared with theoretical ion mass fingerprints of virtually digested and fragmented proteins in the database. Their similarity is determined by a combination of independent scores of correlations between the experimental and theoretical common masses.

Various SPC algorithms have been developed. All are based on a probabilistic score depending on the mass errors and differ mainly by their scoring function, which can be more or less sophisticated. MSTag, PepFrag (Fenyo et al., 1998), and MASCOT (Perkins et al., 1999) are examples. One algorithm—SCOPE (Bafna and Edwards, 2001)—uses both a complex probabilistic model and a dynamic programming method. Another algorithm, SEQUEST (Eng et al., 1994; Yates et al., 1995; Yates et al., 1996; Gatlin et al., 2000), uses two filtering levels: SPC followed by cross-correlation by means of fast Fourier transformation. Concerning modifications, any mutation or PTM of the source protein is susceptible to drastically modify the MS/MS spectra in comparison to the unmodified protein in the reference database: modified fragment masses are shifted by a delta corresponding to the mass difference brought by the modification/mutation. As a result, a source modified peptide might not find any corresponding match in the reference protein database. SPC methods generally include in the database all modified/mutated peptides that they want to consider, which requires prior knowledge of the mass difference associated with the modifications/mutations taken into account. Accordingly, modifications whose mass difference with the unmodified peptide is unpredictable (such as glycosylations) cannot be taken into account by SPC methods. In addition, including all possible modifications/mutations of the peptides in the database is unrealistic due to the combinatorial explosion it implies. As a result, SPC methods usually take into account only a few very common modifications occurring on specific amino acids, such as methionine oxidation or cysteine carbamidomethylation.

Two non-SPC, spectra-matching methods have been described: spectral convolution and spectral alignment, with PEDANTA (Pevzner et al., 2000; Pevzner et al., 2001) their corresponding tool, which deal with modifications/mutations, including unpredictable modifications. However, the number of modifications/mutations considered must be kept sufficiently low in order to allow identifications that are sufficiently discriminating.

As extensively described above, available PMF and MS/MS identification software tools are able to assist with protein identification; however, the final analysis of the results still requires human interpretation and validation. Tools have lately been developed as palliatives to reduce the time required to manually validate the identification, such as Chomper (Eddes et al., 2002) or to measure the quality of those identifications, such as Qscore (Moore et al., 2002). These tools can be very useful for the users decision; however, they do not really reduce the time required for these procedures.

Concerning high throughput purposes, the automated analysis from complete 2-DE gels up to the mass spectra data has been carried out without human intervention (Traini et al., 47; Binz et al., 139). The molecular scanner approach (Binz et al., 139) combines parallel methods for protein digestion and electrotransfer (Bienvenut et al., 141) with peptide mass fingerprinting methods. Massive MS data identifications had to be done automatically, and especially for this case, specific algorithms had to be created to cluster peptide identifications and assign to each spot in a gel a single protein name (Müller et al, 2002).

The recent advances in mass spectrometry automation have brought huge amounts of proteomic mass spectrometry data (PMF or MS/MS) produced every day. High-throughput proteomics projects struggle with the huge amount of mass spectra data that have to be analysed, as one single mass spectrometer is capable of producing as much as one mass spectrum per second and the combination of multiple instruments may produce hundreds or thousands of spectra per minute. As a result, today even the most advanced algorithms for de novo sequencing or spectra matching, operated with the most powerful computers do not suffice to process all incoming PMF or MS/MS data with sufficient rapidity. Therefore, there is a need for a method allowing to identify proteins/peptides without having to analyse each mass spectrum.

### BRIEF DESCRIPTION OF THE INVENTION

This invention relates to an improved method for comparing two or more samples containing proteins. In comparative proteomics, the current approaches require to identify all the proteins present in each tested sample as a first step, before examining which proteins are present, or present in different quantities, in the samples. As a result, an important number of proteins, which are present or present in similar quantities in the samples, are identified several times: once for each sample where a specific protein is present. Accordingly, a significant amount of resources and computing power—that could be better used for other tasks—is basically wasted.

This invention consists in correlating and selecting the huge amount of data generated by mass spectrometry (PMF and MS/MS) before any kind of protein identification process, and then only identifying the proteins that are differentially expressed in dissimilar proteomes and thus have a potentially important biological interest (for example, targets for drug discovery, disease markers, etc.). To do so, the experimental data resulting from separation and mass spectrometry are first correlated according to a correlation method, and then selected according to specific selection criteria. At this stage only, the selected data are analysed to identify the corresponding proteins.

### DESCRIPTION OF THE FIGURE

FIG. 1 is a flow chart of the invention. Two or more protein containing samples (1) are separated by a separating method (2). The resulting separation data (3) are saved for further use. The separated protein content is then cleaved enzymatically during the digestion procedure (4), and mass spectrometry (5) is applied to the resulting peptide mixture. Alternatively, mass spectrometry (5) is applied directly to the separated protein content, without prior enzymatic cleavage. The resulting mass spectrometry data (6) is saved. The experimental data (including the mass spectrometry data (6) and the separation data (3)) for each sample are then correlated with the corresponding experimental data for the other sample or samples, according to a correlation method (7). The correlated data are then selected according to one or more selection criteria (8). The correlation method (7) and selection according to selection criteria (8) are performed through the use of a software (9) especially developed for this task. The proteins corresponding to the selected data are finally identified according to a protein identification method (10). The figure is provided as an example and showing a preferred embodiment of the invention. It is recognized, however, that departures may be made therefrom within the scope of the invention as claimed.

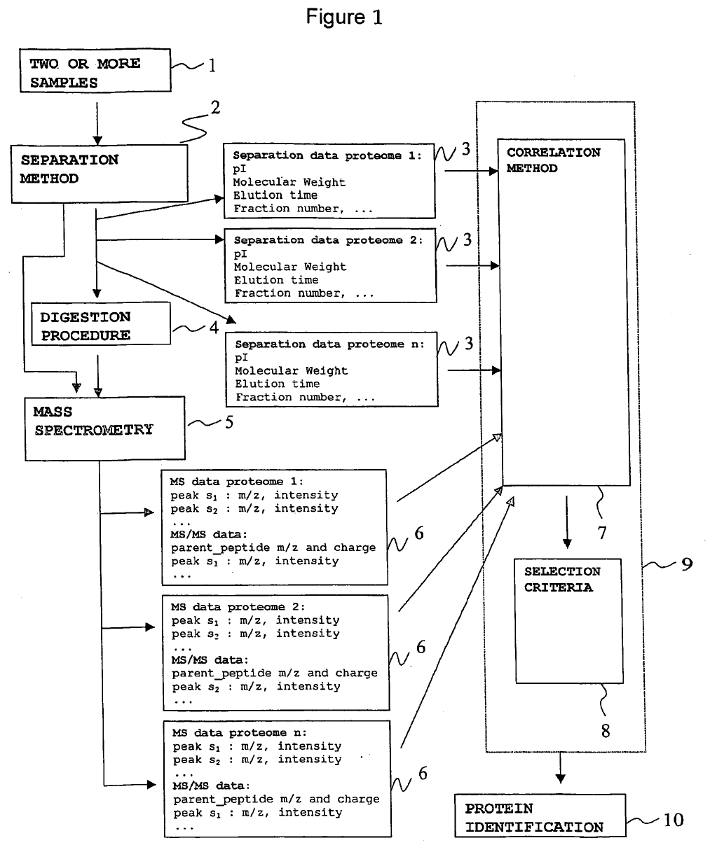

### DESCRIPTION OF THE INVENTION

In order to compare two or more samples containing proteins (1), the present invention comprises the following steps:
a. Providing two or more samples containing proteins (1).
b. Separating each sample by a protein separation method (2) and saving the resulting separation data (3).
c. Cleaving the separated protein content of each sample by enzymatic digestion (4).
d. Analysing the resulting peptide mixture of each sample by mass spectrometry (5) and saving the resulting mass spectrometry data (6).
e. Correlating the experimental data resulting from step (b) and step (d) for each sample with the corresponding experimental data for the other sample or samples, by a correlation method (7).
f. Selecting a subset of the experimental data correlated in step (e) according to one or more of the following selection criteria (8):
   experimental data which is not correlated with any other experimental data in the other sample or samples.
   experimental data which is correlated with experimental data in the other sample or samples, but has a different quantitation.
g. Identifying the proteins corresponding to the experimental data selected in step (f) by a protein identification method (10).

Depending on the nature and size of the proteins resulting from the separation methods, enzymatic digestion (4) according to step (c) may be omitted.

The samples containing proteins (1) according to the invention may be any sample containing proteins such as physiological fluids, proteomes of specific cells, tissues, organisms, etc. Preferably but without limitation, the samples containing proteins (1) are proteomes of similar fluids, tissues or cells in different experimental conditions, environmental conditions or states of development.

The protein separation method (2) according to the invention may use any separation technique known in the art such as but without limitation chromatography, gas chromatography, micro-channel networks, liquid chromatography, high-pressure liquid chromatography, reverse-phase liquid chromatography, electrophoresis, gel electrophoresis, capillary electrophoresis. The protein separation method (2) may also comprise one or more additional rounds of separation using the same separation technique or one or more different separation techniques.

The mass spectrometry (5) according to the invention consists in measuring the masses of peptides or protein fragments, and may be any mass spectrometry technique known in the art such as but without limitation, peptide mass fingerprinting or peptide mass fingerprinting followed by tandem mass spectrometry.

The correlation method (7) according to the invention may comprise one or more of the following methods:
(i) Shared peaks count
(ii) Comparison of column elution times
(iii) Comparison of relative intensities of specific peaks
(iv) Comparison of intensities of specific peaks in relation to an internal or external calibration standard
(v) Spectral alignment
(vi) Clustering algorithms
(vii) Statistical data analysis

The correlation method (7) and selection according to selection criteria (8) may be performed by a computer running software (9) specifically developed for this task, or by any other adequate computational means.

The protein identification method (10) according to the invention consists in any protein identification method known in the art such as but without limitation: matching the selected mass spectrometry data resulting from step (f) above with theoretical mass spectra from a reference protein or nucleic database, de novo sequencing, or de novo sequencing followed by matching of the resulting sequence or sequence tag with a reference protein or nucleic database. The protein identification method (10) may be performed by a computer running software specifically developed for this task, or by any other adequate computational means.

1. A method of comparing two or more samples containing proteins, comprising the following steps:
a. providing two or more samples containing proteins;
b. separating each sample by a protein separation method and saving the resulting separation data;
c. cleaving the separated protein content of each sample by enzymatic digestion;
d. analyzing the resulting peptide mixture of each sample by mass spectrometry and saving the resulting mass spectrometry data;
e. correlating the experimental data resulting from step (b) and step (d) for each sample with the corresponding experimental data for the other sample or samples, by a correlation method;
f. selecting a subset of the experimental data correlated in step (e) according to one or more of the following selection criteria:
experimental data which is not correlated with any other experimental data in the other sample or samples;
experimental data which is correlated with experimental data in the other sample or samples, but has a different quantitation; and
g. identifying the proteins corresponding to the experimental data selected in step (f) by a protein identification method.

2. The method of claim 1, wherein enzymatic digestion according to step (c) is omitted.

3. The method of claim 1, wherein said samples are proteomes of similar physiological fluids, cells, tissues or organisms in different experimental conditions, environmental conditions or states of development.

4. The method of claim 1, wherein said protein separation method uses chromatography as a separation technique.

5. The method of claim 4, wherein said chromatography is gas chromatography, micro-channel networks, liquid chromatography, high-pressure liquid chromatography or reverse-phase liquid chromatography.

6. The method of claim 1, wherein said protein separation method uses electrophoresis as a separation technique.

7. The method of claim 6, wherein said electrophoresis is gel electrophoresis or capillary electrophoresis.

8. The method of claim 1, wherein said protein separation method comprises one or more additional rounds of separation using the same separation technique or one or more different separation techniques.

9. The method of claim 1, wherein said mass spectrometry is peptide mass fingerprinting.

10. The method of claim 1, wherein said mass spectrometry is peptide mass fingerprinting followed by tandem mass spectrometry.

11. The method of any one of claims 1 to 10, wherein said experimental data correlation method comprises one or more of the following methods:
(i) shared peaks counts
(ii) comparison of column elution times;
(iii) comparison of relative intensities of specific peaks;
(iv) comparison of intensities of specific peaks in relation to an internal or external calibration standard;
(v) spectral alignment;
(vi) clustering algorithms;
(vii) statistical data analysis.

12. The method of claim 1, wherein said protein identification method consists in matching the selected mass spectrometry data resulting from step (f) with theoretical mass spectra from a reference protein or nucleic database.

13. The method of claim 1, wherein said protein identification method is de novo sequencing.

14. The method of claim 1, wherein said protein identification method is de novo sequencing followed by matching of the resulting sequence or sequence tag with a reference protein or nucleic database.
